(19) United States
(12) Patent Application Publication
(10) Pub. No.: US 2011/0190603 A1
(43) Pub. Date: Aug. 4, 2011

(54) Sensor Inserter Having Introducer

(76) Inventor: Gary A. Stafford, Hayward, CA (US)

(21) Appl. No.: 12/893,974

(22) Filed: Sep. 29, 2010

Related U.S. Application Data
(60) Provisional application No. 61/246,825, filed on Sep. 29, 2009.

(51) Int. Cl. A61B 5/145

(52) U.S. Cl. 600/309

(57) ABSTRACT

A sensor inserter assembly having an introducer configured with one or more holding members to releasably retain a sensor and/or a compressible member.

### CROSS-REFERENCE TO RELATED APPLICATIONS

This application claims priority to U.S. Provisional Application No. 61/246,825, filed Sep. 29, 2009, which is incorporated by reference in its entirety herein for all purposes.

### FIELD OF THE INVENTION

The present invention relates to a sensor delivery unit. More particularly, the invention relates to a sensor inserter having a safety member to impede actuation of the inserter. The invention also relates to an introducer having a holding member configured to releasably retain a sensor, such as an analyte sensor. The introducer can further comprise a compressible member configured to tent the skin and puncture the skin to a reduced depth during insertion of a sensor. The invention also relates to a method of arming the sensor delivery unit.

### BACKGROUND OF THE INVENTION

Diabetes Mellitus is an incurable chronic disease in which the body does not produce or properly utilize insulin. Insulin is a hormone produced by the pancreas that regulates blood sugar (glucose). In particular, when blood sugar levels rise, e.g., after a meal, insulin lowers the blood sugar levels by facilitating blood glucose to move from the blood into the body cells. Thus, when the pancreas does not produce sufficient insulin (a condition known as Type I Diabetes) or does not properly utilize insulin (a condition known as Type II Diabetes), the blood glucose remains in the blood resulting in hyperglycemia or abnormally high blood sugar levels.

The vast and uncontrolled fluctuations in blood glucose levels in people suffering from diabetes cause long-term, serious complications. Some of these complications include blindness, kidney failure, and nerve damage. Additionally, it is known that diabetes is a factor in accelerating cardiovascular diseases such as atherosclerosis (hardening of the arteries), leading to stroke, coronary heart disease, and other diseases. Accordingly, one important and universal strategy in managing diabetes is to control blood glucose levels.

The first step in managing blood glucose levels is testing and monitoring blood glucose levels by using conventional techniques, such as drawing blood samples, applying the blood to a test strip, and determining the blood glucose level using colorimetric, electrochemical, or photometric test meters. Another more recent technique for monitoring blood glucose levels is by using a continuous or automatic glucose monitoring system, such as for example, the FreeStyle Navigator® Continuous Glucose Monitoring System, manufactured by Abbott Diabetes Care, Inc.

In accordance with the monitoring of blood glucose levels, a sensor is typically subcutaneously or transcutaneously positioned under the skin of a user. In this regard, a sensor inserter assembly, which can be preloaded with a sensor is employed to insert the sensor through the skin of a user. A new sensor is generally implanted under the user's skin every three to seven days. Thus, easy to use sensor inserter assemblies causing reduced trauma to the skin during use are desired.

### SUMMARY

In one embodiment, an introducer is provided that comprises a body portion having a proximal end and a distal end, and a shaft portion including a channel having a distal end. The shaft portion of the introducer can be configured to extend downwardly from an edge of the body portion of the introducer. The shaft portion can include a holding member disposed along a length of the shaft or channel. The holding member can be configured to substantially releasably retain a sensor, or more specifically, a sensor tail inside the introducer channel so that the sensor will be maintained in the introducer prior to insertion, and especially maintain position during shipping, handling, and/or dropping.

In some embodiments, the holding member comprises one or more rolling members disposed along a length of the shaft portion. The rolling members can be configured to contact and releasably retain the sensor. In one embodiment, the rolling members can be configured to rotate. An analyte sensor retained by the shaft portion may be displaced from the shaft portion upon rotation of the rolling members. In some embodiments, the one or more rolling members can be disposed within the channel. In another embodiment, the one or more rolling members are disposed within a sidewall of the channel, such as for example embedding in the wall of the channel. In other embodiments, the holding member comprises a sheath disposed coaxially about the shaft portion. The sheath can comprise one or more perforations along a perforation line disposed along a length of the sheath. In this manner, the sheath can be a tear away member. In some embodiments, the sheath comprises a polymer film. The polymer film can be attached to an outer surface of the shaft portion.

In some embodiments, the shaft portion of the introducer and sensor each comprise either a magnet or a magnetized area such that the sensor is releasably retained by the introducer by magnetic forces. In this regard, in accordance with one embodiment, magnetic material can be embedded on the surface of the sensor. Further, a magnet or a magnetized area is fit into the sharp to hold the sensor in place. Release of the magnetic force an occur when the sharp is removed as part of the insertion process of the sensor delivery unit.

In another embodiment, the shaft portion can include one or more windings configured to releasably retain the sensor. The sensor can be displaced from the shaft portion by unwinding the one or more windings. In some embodiments, the windings comprise wound rolls of polymer film.

In yet another embodiment, the introducer shaft portion can be configured to include an elongate slot formed in the channel. The sensor can include a flange or a pin to be disposed within or engage the channel. In some embodiment, the flange or pin extends from an edge of the sensor and protrudes through the slot. In some embodiment, the slot includes a distal section configured to be wider than the width of a proximal section, and sufficiently wide such that the sensor flange or pin may be displaced from the shaft when the flange becomes disposed in the wider section of the slot, for example during the insertion process as the sensor travels towards an insertion position. In other embodiments, the shaft can include a diaphragm, such as a thin, semi-rigid membrane housed along a portion of the channel. The diaphragm can include an opening to receive and retain the sensor. Alternatively, the holding member can include a sponge material disposed along the channel of the shaft portion. The sponge material can be configured to provide a soft interference fit with a sensor disposed in the shaft portion.

In another aspect of the invention, the introducer can include a compressible member attached to a lateral side of the shaft portion. The compressible body includes a distal end that extends distal to the distal end of the shaft portion. During operation, the distal end of the compressible body contacts and can tent or provide a depression in the skin, further upon compression of the member, the introducer sharp can penetrate skin of a user.

In some embodiment, the compressible member can include a first section and a second section. The first section can include a compressible body. For example, the compressible body can include a spring. The second section of the compressible member can be non-compressible, but retractable.

In another aspect of the invention, a sensor insertion assembly having a safety member is provided. The safety member is configured to engage the actuator such that actuation is impeded. The safety member includes a member having a body disposed through an aperture or a slot formed in the actuator body. For example but not limitation, the safety member can include for example, a pin member, D-ring, press-clip, plug. The safety member can include a pull tab for removal of safety member. In some embodiments, they safety member is formed from thermoplastic material.

In another aspect of the invention, a method is provided to arm the sensor inserter assembly. In this regard, a safety member is not necessary. In this embodiment, the inserter assembly includes a pin that contacts a carrier connected to the introducer sharp. The carrier moves latching arms into a locked position to arm the inserter by contacting the distal end of the pin with a surface. The pin can then be removed from the inserter for use.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a perspective view showing a sensor inserter and adhesive mount constructed in accordance with the disclosed subject matter;

FIG. 2 is a perspective view of the adhesive mount and sensor attached to the user's skin in accordance with the disclosed subject matter;

FIG. 3 is a perspective view of the transmitter attached to the adhesive mount in accordance with the disclosed subject matter;

FIG. 4 is an exploded perspective view of the embodiment of FIG. 1;

FIG. 5 is a cross-sectional view of the inserter of FIG. 1;

FIG. 6 is a schematic depiction of an introducer and sensor in accordance with the disclosed subject matter;

FIG. 7 is a is a schematic depiction of an shaft portion of the introducer in accordance with the disclosed subject matter;

FIG. 8 is a perspective view of a shaft portions having one or more holding member in accordance with the disclosed subject matter;

FIG. 9 is a sectional view of the shaft portion of FIG. 8 in accordance with the disclosed subject matter;

FIG. 10 is a sectional view of a shaft portions having one or more holding member in accordance with the disclosed subject matter;

FIG. 11 is a perspective view of the shaft portion of FIG. 10 in accordance with the disclosed subject matter;

FIG. 12 is a perspective view of a shaft portions having one or more holding members in accordance with the disclosed subject matter;

FIG. 13 is a sectional view of a shaft portions having one or more holding members in accordance with the disclosed subject matter in a first configuration;

FIG. 14 is a sectional view of a shaft portions having one or more holding member in accordance with the disclosed subject matter in a second configuration;

FIG. 15 is a sectional view of a shaft portions having one or more holding member in accordance with the disclosed subject matter in a second configuration;

FIG. 16 is a perspective view of a shaft portions of FIG. 15 in accordance with the disclosed subject matter;

FIG. 17 is a perspective view of a shaft portions of FIG. 15 in accordance with the disclosed subject matter;

FIGS. 18-20 are views of a shaft portions in accordance with the disclosed subject matter;

FIG. 21 is a sectional view of a shaft portions having one or more holding member in accordance with the disclosed subject matter in a second configuration;

FIG. 22 is a sectional view of a shaft portion having one or more holding member in accordance with the disclosed subject matter in a second configuration;

FIG. 23 is a side view of a shaft portion comprising a compressible member in accordance with the disclosed subject matter in a second configuration;

FIG. 24 is an enlarged side view of a shaft portion of FIG. 23 in accordance with the disclosed subject matter in a second configuration;

FIG. 25 is a partial side view of a shaft portion of FIG. 23 in accordance with the disclosed subject matter in a second configuration;

FIG. 26 is a sectional view of a shaft portion of FIG. 23 in accordance with the disclosed subject matter in a second configuration;

FIG. 27 is a side view of a shaft portion of FIG. 23 in accordance with the disclosed subject matter in a second configuration;

FIG. 28-30 are schematic depictions the introducer of FIGS. 23-27 depressing the skin and retracting to allow introducer sharp to pierce the skin in accordance with the disclosed subject matter;

FIGS. 31-45 are perspective views of some embodiments of the safety member of the sensor inserter assembly in accordance with the disclosed subject matter;

FIG. 46 is a cross-sectional view of an inserter having a pin disposed against the shuttle of the inserter in accordance with the disclosed subject matter; and

FIGS. 47-49 illustrate a method of arming a sensor.

### DETAILED DESCRIPTION OF EXEMPLARY EMBODIMENTS

A detailed description of the disclosure is provided herein. It should be understood, in connection with the following description, that the subject matter is not limited to particular embodiments described, as the particular embodiments of the subject matter may of course vary. It is also to be understood that the terminology used herein is for the purpose of describing particular embodiments only, and is not intended to be limiting, since the scope of the disclosed subject matter will be limited only by the appended claims.

Where a range of values is provided, it is understood that each intervening value between the upper and lower limit of that range and any other stated or intervening value in that stated range, is encompassed within the disclosed subject matter. Every range stated is also intended to specifically disclose each and every “subrange” of the stated range. That is, each and every range smaller than the outside range specified by the outside upper and outside lower limits given for a range, whose upper and lower limits are within the range from said outside lower limit to said outside upper limit (unless the context clearly dictates otherwise), is also to be understood as encompassed within the disclosed subject matter, subject to any specifically excluded range or limit within the stated range. Where a range is stated by specifying one or both of an upper and lower limit, ranges excluding either or both of those stated limits, or including one or both of them, are also encompassed within the disclosed subject matter, regardless of whether or not words such as “from”, “to”, “through”, or “including” are or are not used in describing the range.

Unless defined otherwise, all technical and scientific terms used herein have the same meaning as commonly understood by one of ordinary skill in the art to which this disclosed subject matter belongs. Although any methods and materials similar or equivalent to those described herein can also be used in the practice or testing of the present disclosed subject matter, this disclosure may specifically mention certain exemplary methods and materials.

All publications mentioned in this disclosure are, unless otherwise specified, incorporated herein incorporated by reference in its entirety herein for all purposes, including without limitation to disclose and describe the methods and/or materials in connection with which the publications are cited.

The publications discussed herein are provided solely for their disclosure prior to the filing date of the present application. Nothing herein is to be construed as an admission that the present disclosed subject matter is not entitled to antedate such publication by virtue of prior invention. Further, the dates of publication provided may be different from the actual publication dates, which may need to be independently confirmed.

As used herein and in the appended claims, the singular forms “a”, “an”, and “the” include plural referents unless the context clearly dictates otherwise.

Nothing contained in the Abstract or the Summary should be understood as limiting the scope of the disclosure. The Abstract and the Summary are provided for bibliographic and convenience purposes and due to their formats and purposes should not be considered comprehensive.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosed subject matter. Any recited method can be carried out in the order of events recited, or in any other order which is logically possible. Reference to a singular item, includes the possibility that there are plural of the same item present. When two or more items (for example, elements or processes) are referenced by an alternative “or”, this indicates that either could be present separately or any combination of them could be present together except where the presence of one necessarily excludes the other or others.

Various exemplary embodiments of the analyte monitoring system and methods of the invention are described in further detail below. Although the invention is described primarily with respect to a glucose monitoring system, each aspect of the invention is not intended to be limited to the particular embodiment so described. Accordingly, it is to be understood that such description should not be construed to limit the scope of the invention, and it is to be understood that the analyte monitoring system can be configured to monitor a variety of analytes, as described below. Further, section headers, where provided, are merely for the convenience of the reader and are not to be taken as limiting the scope of the invention in any way, as it will be understood that certain elements and features of the invention have more than one function and that aspects of the invention and particular elements are described throughout the specification.

### A. Overview

The invention is generally directed to an analyte monitoring system including an apparatus, such as an inserter, configured to insert various devices into the body of a subject, such as for example, an analyte sensor, an infusion set, or a lancing device.

Certain classes of analyte monitoring systems are provided in small, lightweight, battery-powered and electronically-controlled systems. Such systems may be configured to detect signals indicative of in vivo analyte levels using an electrochemical sensor, and collect such signals, with or without processing the signal. In some embodiments, the portion of the system that performs this initial processing may be configured to provide the raw or initially processed data to another unit for further collection and/or processing. Such provision of data may be effected, for example, via a wired connection, such as an electrical, or via a wireless connection, such as an IR or RF connection.

Certain analyte monitoring systems for in vivo measurement employ a sensor that measures analyte levels in interstitial fluids under the surface of the subject's skin. These may be inserted partially through the skin or positioned entirely under the skin. A sensor in such a system may operate as an electrochemical cell. Such a sensor may use any of a variety of electrode configurations, such as a three-electrode configuration (e.g., with “working”, “reference” and “counter” electrodes), driven by a controlled potential (potentiostat) analog circuit, a two-electrode system configuration (e.g., with only working and counter electrodes), which may be self-biasing and/or self-powered, and/or other configurations. In some embodiments, the sensor may be positioned within a blood vessel.

In certain systems, the analyte sensor is in communication with a sensor control unit. As used in this disclosure, an on-body unit sometimes refers to such a combination of an analyte sensor with such a sensor control unit. The analyte monitoring system may include an on-body unit including a sensor and a monitor unit.

In some embodiments, the on-body unit includes electronics configured to process the signal generated by the sensor and may further include a transmitter, transceiver, or other communications electronics to provide the processed data to the monitor unit via a communication link between the on-body unit and the monitor unit.

The monitor unit can include a display for displaying or communicating information to the user of the analyte monitoring system or the user's health care provider or another. The monitor unit is also referred to in this disclosure as a “receiver unit” or “receiver device”, or in some contexts, depending on the usage, as a “display unit,” “handheld unit,” or “meter”. In some embodiments, receiver may also include buttons and/or scroll wheel which allow a user to interact with a user interface located on receiver. The monitor unit, in some embodiments, may include, e.g., a mobile telephone device, a personal digital assistant, other consumer electronic device such as MP3 device, camera, radio, etc., or other communication-enabled data processing device.

The monitor unit may perform data processing and/or analysis, etc. on the received analyte data to generate information pertaining to the monitored analyte levels. The monitor unit may incorporate a display screen, which can be used, for example, to display measured analyte levels, and/or audio component such as a speaker to audibly provide information to a user, and/or a vibration device to provide tactile feedback to a user. It is also useful for a user of an analyte monitor to be able to see trend indications (including the magnitude and direction of any ongoing trend), and such data may be displayed as well, either numerically, or by a visual indicator, such as an arrow that may vary in visual attributes, such as size, shape, color, animation, or direction. The receiver device may further incorporate an in vitro analyte test strip port and related electronics in order to be able to make discrete (e.g., blood glucose) measurements.

In the embodiment described herein, on-body unit and monitor unit communicate via communications link (in this embodiment, a wireless RF connection). Communication may occur, e.g., via RF communication, infrared communication, Bluetooth communication, Zigbee communication, 802.1 x communication, or WiFi communication, etc., In some embodiments, the communication may include an RF frequency of 433 MHz, 13.56 MHz, or the like. In some embodiments, a secondary monitor unit may be provided. A data processing terminal may be provided for providing further processing or review of analyte data.

In certain embodiments, the analyte monitoring system may be a continuous analyte monitor (e.g., a continuous glucose monitoring system or CGM), and accordingly operate in a mode in which the communications via communications link has sufficient range to support a flow of data from the on-body unit to the monitor unit. In some embodiments, the data flow in a CGM system is automatically provided by the on-body unit to the monitor unit. For example, in some embodiments no user intervention is required for the on-body unit to send the data to the monitor unit. In some embodiments, the on-body unit provides the signal relating to analyte level to the receiving unit 300 on a periodic basis. For example, the signal may be provided, e.g., automatically sent, on a fixed schedule, e.g., once every 250 ms, once a second, once a minute, etc. In some embodiments, the signal is provided to the monitor unit upon the occurrence of an event, e.g., a hyperglycemic event or a hypoglycemic event, etc. In some embodiments, on-body unit may further include local memory in which it may record “logged data” or buffered data collected over a period of time and provide the some or all of the accumulated data to monitor unit from time-to-time. A separate data logging unit may be provided to acquire periodically received data from on-body unit. Data transmission may be one-way communication, e.g., the on-body unit provides data to the monitor unit without receiving signals from the monitor unit. In some embodiments, two-way communication is provided between the on-body unit and the monitor unit.

In some embodiments, the analyte monitoring system includes a sensor which obtains an analyte signal which is provided to the monitor unit “on demand.” According to such embodiments, the monitor unit requests a signal from the on-body unit, or the on-body unit may be activated to send signal upon activation to do so. Accordingly, one or both of the on-body unit and monitor unit may include a switch activatable by a user or activated upon some other action or event, the activation of which causes analyte-related signal to be transferred from the on-body unit to the monitor unit. For example, the monitor unit is placed in close proximity with a transmitter device and initiates a data transfer, either over a wired connection, or wirelessly by various means, including, for example various RF-carried encodings and protocols and IR links.

In some embodiments, the signal relating to analyte level is instantaneously generated by the analyte sensor upon receipt of the request, and provided to the monitor unit as requested, and/or the signal relating to analyte level is periodically obtained, e.g., once every 250 ms, once a second, once a minute, etc. Upon receipt of the “on demand” request at the on-body unit, an analyte signal is provided to the monitor unit. In some cases, the signal provided to the monitor unit is or at least includes the most recent analyte signal(s).

In further embodiments, additional data is provided to the monitor unit “on demand.” For example, analyte trend data may be provided. Such trend data may include two or more analyte data points to indicate that analyte levels are rising, falling, or stable. Analyte trend data may include data from longer periods of time, such as, e.g., several minutes, several hours, several days, or several weeks.

Further details regarding on demand systems are disclosed in U.S. Pat. No. 7,620,438, U.S. Patent Publication Nos. 2009/0054749 A1, published Feb. 26, 2009; 2007/0149873 A1, published Jun. 28, 2007; 2008/0064937 A1, published Mar. 13, 2008; 2008/0071157 A1, published Mar. 20, 2008; 2008/0071158 A1, published Mar. 20, 2008; 2009/0281406 A1, published Nov. 12, 2009; 2008/0058625 A1, published Mar. 6, 2008; 2009/0294277 A1, published Dec. 3, 2009; 2008/0319295 A1, published Dec. 25, 2008; 2008/0319296 A1, published Dec. 25, 2008; 2009/0257911 A1, published Oct. 15, 2009, 2008/0179187 A1, published Jul. 31, 2008; 2007/0149875 A1, published Jun. 28, 2007; 2009/0018425 A1, published Jan. 15, 2009; and pending U.S. patent application Ser. No. 12/625,524, filed Nov. 24, 2009; Ser. No. 12/625,525, filed Nov. 24, 2009; Ser. No. 12/625,528, filed Nov. 24, 2009; Ser. No. 12/628,201, filed Nov. 30, 2009; Ser. No. 12/628,177, filed Nov. 30, 2009; Ser. No. 12/628,198, filed Nov. 30, 2009; Ser. No. 12/628,203, filed Nov. 30, 2009; Ser. No. 12/628,210, filed Nov. 30, 2009; Ser. No. 12/393,921, filed Feb. 27, 2009; 61/149,639, filed Feb. 3, 2009; Ser. No. 12/495,709, filed Jun. 30, 2009; 61/155,889, filed Feb. 26, 2009; 61/155,891, filed Feb. 26, 2009; 61/155,893, filed Feb. 26, 2009; 61/165,499, filed Mar. 31, 2009; 61/227,967, filed Jul. 23, 2009; 61/163,006, filed Mar. 23, 2009; Ser. No. 12/495,730, filed Jun. 30, 2009; Ser. No. 12/495,712, filed Jun. 30, 2009; 61/238,461, filed Aug. 31, 2009; 61/256,925, filed Oct. 30, 2009; 61/238,494, filed Aug. 31, 2009; 61/238,159, filed Aug. 29, 2009; 61/238,483, filed Aug. 31, 2009; 61/238,581, filed Aug. 31, 2009; 61/247,508, filed Sep. 30, 2009; 61/247,516, filed Sep. 30, 2009; 61/247,514, filed Sep. 30, 2009; 61/247,519, filed Sep. 30, 2009; 61/249,535, filed Oct. 7, 2009; Ser. No. 12/544,061, filed Aug. 19, 2009; Ser. No. 12/625,185, filed Nov. 24, 2009; Ser. No. 12/625,208, filed Nov. 24, 2009; Ser. No. 12/624,767, filed Nov. 24, 2009; Ser. No. 12/242,780, filed Sep. 30, 2008; Ser. No. 12/183,602, filed Jul. 31, 2008; Ser. No. 12/211,014, filed Sep. 15, 2008; and Ser. No. 12/114,359, filed May 2, 2008, each of which is incorporated by reference in its entirety herein for all purposes.

### B. Sensor

The sensor, in accordance with one embodiment of the invention, can be configured to detect and monitor an analyte of interest present in a biological sample of a user. The biological sample can be a biological fluid containing the analyte of interest, such as (but not limited to) interstitial fluid, blood, and urine. The analyte of interest can be one or more analytes including acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. However, other suitable analytes can also be monitored, as would be known in the art. Furthermore, the analyte monitoring system can be configured to monitor the concentration of drugs, such as, for example, antibiotics (e.g., gentamicin, vancomycin, and the like), digitoxin, digoxin, theophylline, warfarin, and the like.

During use, the sensor is physically positioned in or on the body of a user whose analyte level is being monitored by an insertion device. The sensor can be configured to continuously sample the analyte level of the user and convert the sampled analyte level into a corresponding data signal for transmission by the transmitter. In some embodiments, the sensor is implantable into a subject's body for a period of time (e.g., three days, five days, seven days, etc.) to contact and monitor an analyte present in the biological fluid. Thus, a new sensor must be used typically every three to seven days.

Generally, the sensor comprises a substrate, one or more electrodes, a sensing layer and a barrier layer, as described below and disclosed in U.S. Pat. Nos. 6,284,478 and 6,990,366, the disclosures of which are incorporated by reference in their entirety herein for all purposes. In one embodiment, the sensor includes a substrate. In some embodiments, the substrate is formed from a relatively flexible material. Suitable materials for a flexible substrate include, for example, non-conducting plastic or polymeric materials and other non-conducting, flexible, deformable materials. Suitable plastic or polymeric materials include thermoplastics such as polycarbonates, polyesters (e.g., Mylar® and polyethylene terephthalate (PET)), polyvinyl chloride (PVC), polyurethanes, polyethers, polyamides, polyimides, or copolymers of these thermoplastics, such as PETG (glycol-modified polyethylene terephthalate). In other embodiments, the sensor includes a relatively rigid substrate. Suitable examples of rigid materials that may be used to form the substrate include poorly conducting ceramics, such as aluminum oxide and silicon dioxide. Further, the substrate can be formed from an insulating material. Suitable insulating materials include polyurethane, Teflon (fluorinated polymers), polyethyleneterephthalate (PET, Dacron) or polyimide.

The substrate 110 can include a distal end and a proximal end. In some embodiments, the distal and proximal ends have different widths. In some embodiments, the distal and proximal ends have the same width. In some embodiments, the proximal end of the sensor remains above the skin surface. In such embodiments, the distal end of the substrate may have a relatively narrow width. Moreover, sensors intended to be positioned at least partially into the tissue of a user's body at can be configured to have narrow distal end or distal point to facilitate the insertion of the sensor. For example, for insertable sensors designed for continuous or periodic monitoring of the analyte during normal activities of the patient, a distal end of the sensor which is to be implanted into the user has a width of 2 mm or less, preferably 1 mm or less, and more preferably 0.5 mm or less.

A plurality of electrodes can be disposed near the distal end of sensor. The electrodes include working electrode, counter electrode and reference electrode. Other embodiments, however, can include a greater or fewer number of electrodes.

Each of the electrodes is formed from conductive material, for example, a non-corroding metal or carbon wire. Suitable conductive materials include, for example, vitreous carbon, graphite, silver, silver-chloride, platinum, palladium, or gold. The conductive material can be applied to the substrate by various techniques including laser ablation, printing, etching, silk-screening, and photolithography. In one embodiment, each of the electrodes is formed from gold by a laser ablation technique. The sensor can include conductive traces extending from electrodes to corresponding, respective contacts to define the sensor electronic circuitry. In one embodiment, an insulating substrate (e.g., dielectric material) and electrodes are arranged in a stacked orientation (i.e., insulating substrate disposed between electrodes). Alternatively, the electrodes can be arranged in a side by side orientation (not shown), as described in U.S. Pat. No. 6,175,752, the disclosure of which is incorporated by reference in its entirety herein for all purposes.

The sensor can include a sensing material having one or more components designed to facilitate the electrolysis of the analyte of interest. The components, for example, may be immobilized on the working electrode. Alternatively, the components of the sensing layer may be immobilized within or between one or more membranes or films disposed over the working electrode or the components may be immobilized in a polymeric or sol-gel matrix. Further aspects of the sensor is described in U.S. Pat. Nos. 5,262,035, 5,264,104, 5,264,105, 5,320,725, 5,593,852, and 5,665,222, each of which is incorporated by reference in its entirety herein for all purposes.

In some embodiments, the sensor is a self-powered analyte sensor, which is capable of spontaneously passing a currently directly proportional to analyte concentration in the absence of an external power source. Any exemplary sensor is described in U.S. application Ser. No. 12/393,921, filed Feb. 26, 2009, entitled “Self-Powered Analyte Sensor,” which is hereby incorporated by reference in its entirety herein for all purposes.

### C. Inserter

In one aspect of the invention, an inserter is provided. The object to be inserted into the subject can be, for example, an analyte sensor as described above. Alternatively, other objects such as but not limited to an infusion set, or lancing device can be inserted.

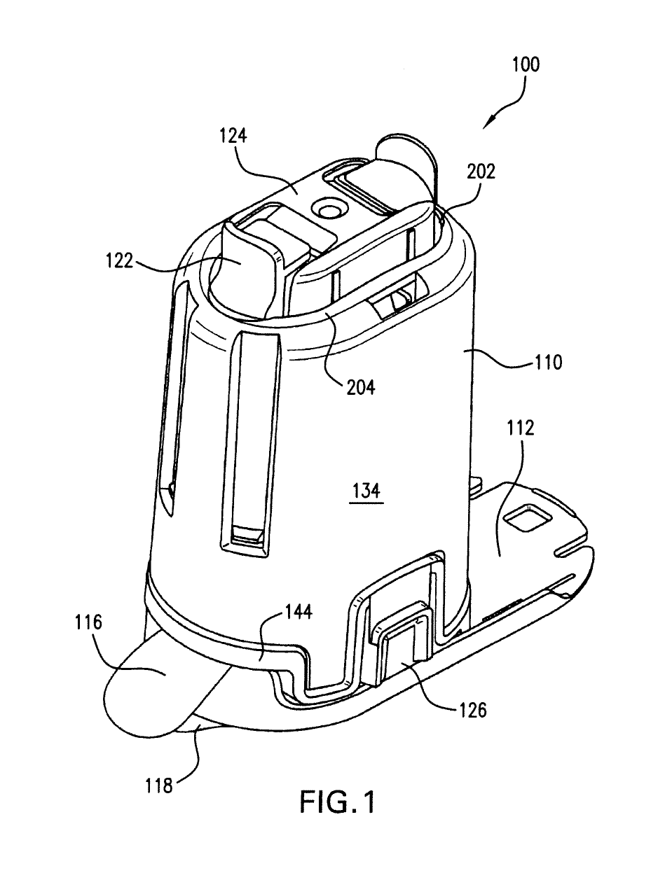

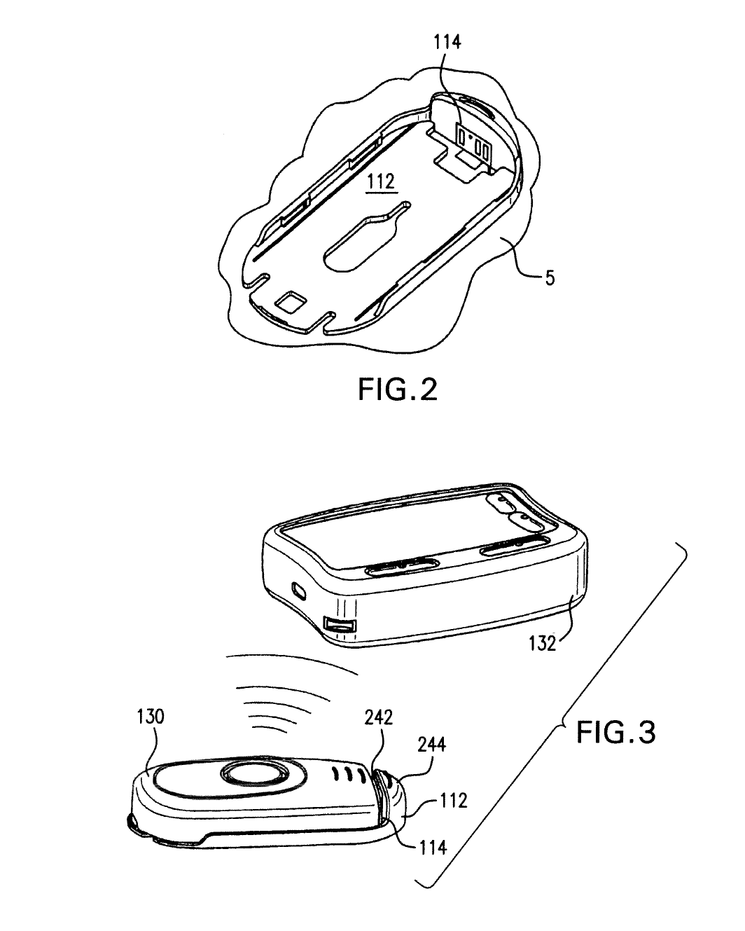

An exemplary embodiment of the sensor inserter assembly 100 is illustrated in FIGS. 1-5. Generally, the sensor inserter assembly 100 includes a sensor (not shown) preloaded within inserter 110. After preparing an insertion site on the skin of a user, the user removes an upper liner 116 and lower liner 118 from an adhesive mount 112 to expose the bottom surface and a portion of the top surface of an adhesive tape located on the bottom surface of the mount 112. Mount 112, with inserter 110 attached, is then applied to the user's skin at the insertion site. The inserter includes an actuator button 124 to be pressed causing inserter 110 to fire, thereby inserting sensor 114 (not shown in FIG. 1) into the user's skin S. In some embodiments of the invention, the inserter 110 includes a safety member to impede actuation of the inserter as described below.

Once sensor 114 has been inserted into the skin S, the user removes inserter 110 from mount 112 by pressing release tabs 126 on opposite sides of inserter 110 and lifting inserter 110 away from mount 112. Further details of the inserter assembly 100 are provided in U.S. Pat. No. 7,381,184, which is incorporated by reference in its entirety herein for all purposes.

Once inserter 110 is removed from mount 112, sensor electronics unit 130 can be slid into place, as illustrated in FIG. 3. The circuitry of sensor electronics unit 130 makes electrical contact with the contacts on sensor 114 after sensor electronics unit 130 is fully seated on mount 112. As discussed hereinabove, mount 112, together with sensor 114, and sensor electronics unit 113 comprises an on-body unit. In some embodiments, sensor electronics unit 130 may include communications circuitry, such as a transmitter, transceiver, or the like, for communicating with additional equipment. For example, once initialization and synchronization procedures are completed, electrochemical measurements from sensor 114 can be sent, e.g., wirelessly from sensor electronics unit 113 to a monitor unit, such as portable receiver 132, as shown in FIG. 3. Sensor 114, mount 112 and sensor electronics unit 130 remain in place on the user for a predetermined period, currently envisioned to be several hours, to several days, e.g., about three days, about five days, about seven days, etc. After expiration of the lifetime of the sensor, these components are then removed so that sensor 114 and mount 112 can be properly discarded. The entire procedure above can then be repeated with a new inserter 110, sensor 314 and mount 112. In some embodiments, the sensor electronics unit 130 and receiver 132 are durable and are reused.

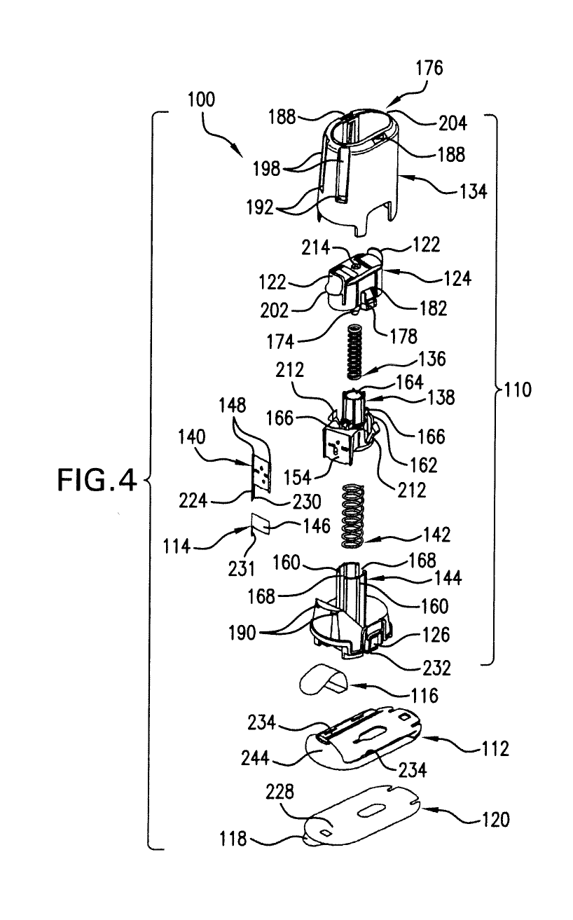

Referring to FIG. 4, the inserter assembly 100 according to one embodiment can be assembled as shown from the following components: e.g., housing 134, actuator button 124, drive spring 136, shuttle 138, introducer sharp 140, sensor 114, retraction spring 142, inserter base 144, upper liner 116, mounting unit 112, adhesive tape 120, and lower liner 118.

Sensor 114 has a main surface 146 slidably mounted between U-shaped rails 148 of introducer sharp 140. Introducer sharp 140 can be mounted to face 154 of shuttle 138, such as with adhesive, heat stake or ultrasonic weld.

In some embodiments, shuttle 138 can be slidably and non-rotabably constrained on base 144 by arcuate guides 160. The shuttle can be generally formed by an outer ring 162 and an inner cup-shaped post 164 connected by two bridges 166. Bridges 166 can be configured to slide between the two slots 168 formed between guides 160 and allow shuttle 138 to travel along guides 160 without rotating. Retraction spring 142 can be captivated at its outer circumference by guides 160, at its bottom by the floor 170 of base 144, at its top by bridges 166, and at its inner circumference by the outer surface of shuttle post 164. Drive spring 136 is captivated at its bottom and outer circumference by the inside surface of shuttle post 164, at its top by the ceiling 172 inside actuator button 124, and at its inner circumference by stem 174 depending from ceiling 172.

When drive spring 136 is compressed between actuator button 124 and shuttle 138 it can urge shuttle 138 towards base 144. When retraction spring 142 is compressed between shuttle 138 and base 144, it urges shuttle 138 towards actuator button 124.

In some embodiments, the actuator button 124 is slidably received within housing 134 from below and resides in opening 176 at the top of housing 134 with limited longitudinal movement. Arms 178 on each side of actuator button 124 can be configured to travel in channels 180 along the inside walls of housing 134, as best seen in FIG. 10. Longitudinal movement of actuator button 124 can be limited in one direction by the base 182 of arms 178 contacting the edge of opening 176 at the top of housing 134, and in the other direction by the distal ends 184 of arms 178 contacting stops 186 in channels 180. Slots 188 are preferably provided in the top of housing 134 for ease of housing manufacture and so tools can be inserted to inwardly compress arms 178 beyond stops 186 to allow actuator button 124 to be removed from housing 134 if needed.

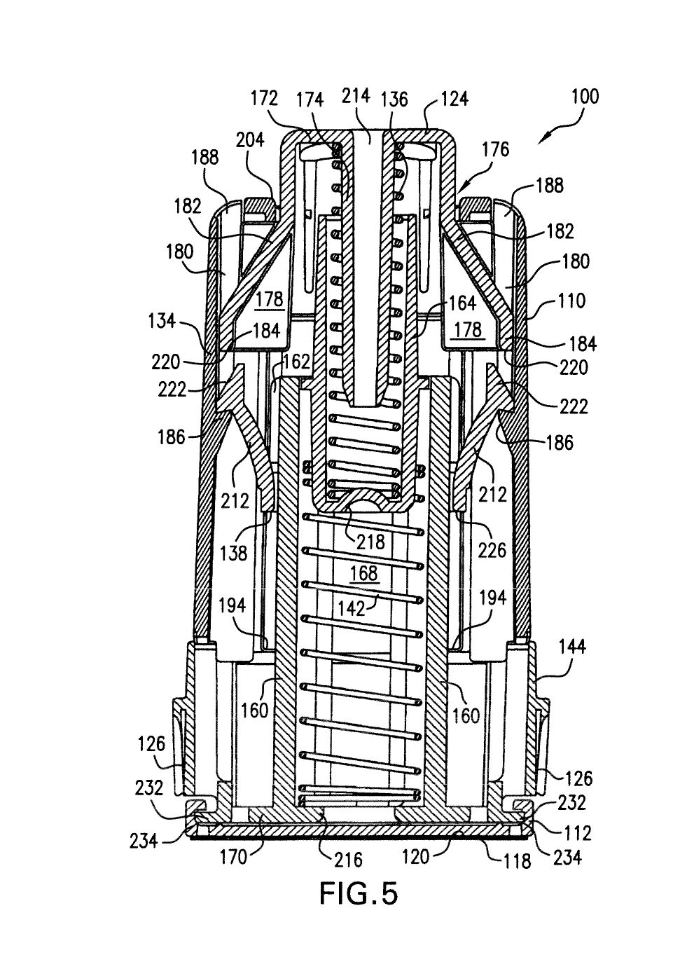

When sensor 114, introducer 140, shuttle 138, retraction spring 142, drive spring 136 and actuator button 124 are assembled between base 144 and housing 134 as shown in FIG. 5 and described above, housing 134 is snapped into place on base 144. Base 144 is held onto housing 134 by upper base barbs 190 that engage upper openings 192 in housing 134, and lower base barbs 194 that engage lower openings 192 in housing 134.

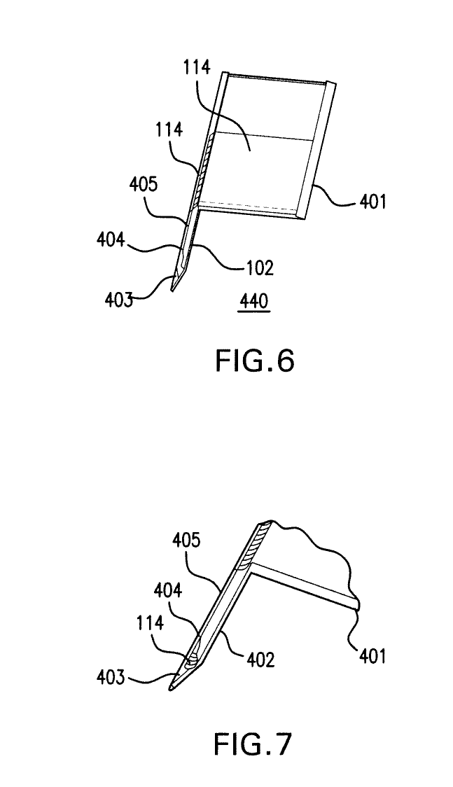

Generally, in accordance with one embodiment of the invention, as illustrated in FIG. 6, an introducer 440 is provided which comprises a body portion 401 and a shaft portion 405. Introducer 440 is substantially identical to introducer 140, and useful with an inserter, such as inserter assembly 100 described hereinabove, with the differences illustrated in the accompanying figures, and described herein. The shaft portion 405 can include a substantially sharp distal edge segment 403 to contact and pierce the skin of a user for transcutaneous placement of the sensor through the user's skin S. As shown, the sensor 114 is retained within the shaft portion 405 of the introducer 440 and is configured to be held in position during insertion of the sensor through the user's skin by the substantially hollow cylindrical shape of the shaft portion 405, as illustrated in FIG. 6.

In some embodiments, referring to FIGS. 6 and 7, the tip of the analyte sensor 114 can be retained at the distal edge segment 403 of the introducer 440 during the subcutaneously or transcutaneous positioning of the sensor 114 through the user's skin. Thus, the sensor 114 is positioned within the substantially hollow shaft portion 405 of the introducer 440. The distal edge segment 403 of the introducer 440 is configured to first pierce through the user's skin, and guide sensor retained in the shaft portion 405 of the introducer 440 through the pierced skin of the user. After placement of the sensor 114 at the desired location under the skin, the introducer 440 can retracted from the user, leaving the sensor 114 in place. In some embodiments, during the introducer removal process, a radial configuration 404 of the shaft portion 405 is configured to guide the removal of the introducer 440 from the pierced skin.

In some embodiments, the shaft portion includes one or more holding members configured to retain the sensor in the introducer. For example but not limitation, the shaft portion 405 of the introducer 440 may have a ribbed configuration to provide additional friction fit during the insertion the introducer and sensor through the skin of the user.

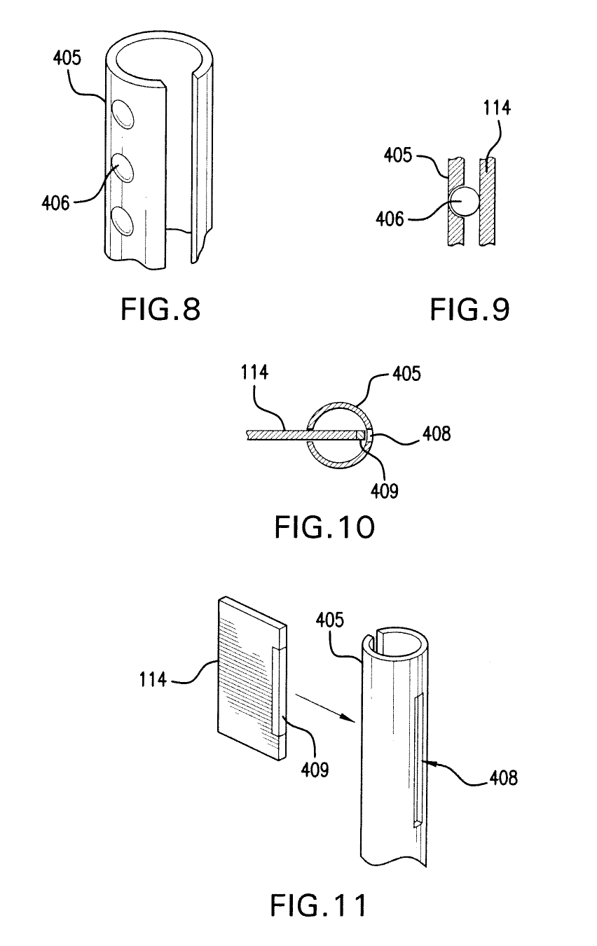

The holding member can include various configurations, as depicted in FIGS. 8 to 31. In one embodiment, as shown in FIGS. 8-9, the shaft portion 405 may include one or more rolling members 406. The rolling members 406 can include for example rollers, balls, or wheels. In some embodiments, the rolling members 406 are disposed within the channel or wall of the shaft portion 405. The rolling members 406 are configured to retain the sensor 114 in the introducer 140 by friction forces prior to insertion of the sensor 114 into the user's body. During the insertion process, the rolling members 406 can turn or rotate to displace the sensor 114 from the introducer shaft 405 during the insertion process. When the sensor 114 is placed at the desired depth and caught in the mount as part of the insertion (e.g., by hook, clamp or gripper), the rolling members 406 rotate from the friction from the sensor 114 as the introducer exits back into the inserter.

In some embodiments, as shown in FIGS. 10-11, the shaft portion 405 of the introducer 140 and the sensor 114 comprise a magnet 408 or magnetized area 409, such that magnetic forces retain the sensor within the introducer. The magnetic material can be any material that will provide magnetic forces including but not limited to low grade stainless steel, carbon ink, and the like. In some embodiments, the shaft or the sensor can be doped with magnetic metal. The magnet can be disposed along the channel of the shaft portion. In this regard, in accordance with one embodiment, magnetic material can be embedded on the surface of the sensor. Further, a magnet or a magnetized area is fit into the sharp to hold the sensor in place. Release of the magnetic force an occur when the shaft portion 105 is removed as part of the insertion process of the sensor delivery unit.

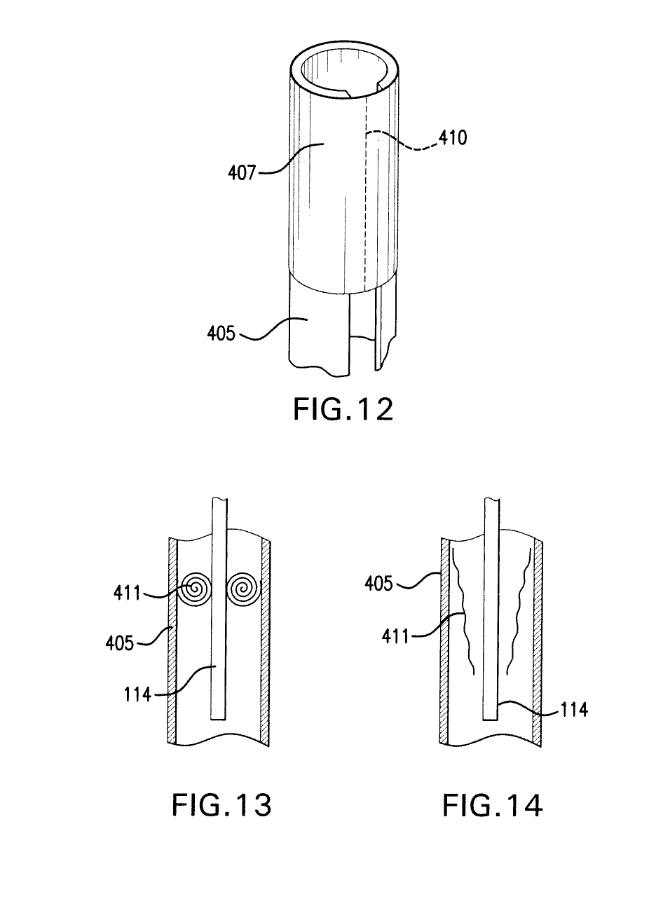

In other embodiments, as illustrated in FIG. 12, the holding member comprises a sheath 407 disposed coaxially about the shaft portion 405. The sheath 407 can comprise one or more perforations along a perforation line 410 disposed along a length of the sheath. In this manner, the sheath can be a tear away member. In some embodiments, the sheath comprises a polymer film. The polymer film can be attached to an outer surface of the shaft portion. Suitable materials for the sheath include polyimide, Pebax, polyethylene, Nylon, PTFE, polyester, and polyurethane.

In another embodiment, as depicted in FIGS. 13-14, the shaft portion 405 can include one or more windings 411 configured to releasably retain the sensor 114. The windings are generally a wound member 411 having the capability to unwind, as illustrated in FIG. 14. While the winding 411 is in the wound configuration, it applies an interference against the sensor body to retain the sensor 114. The sensor can be displaced from the shaft portion 405 upon unwinding the one or more windings. In some embodiments, the windings comprise wound rolls of polymer film.

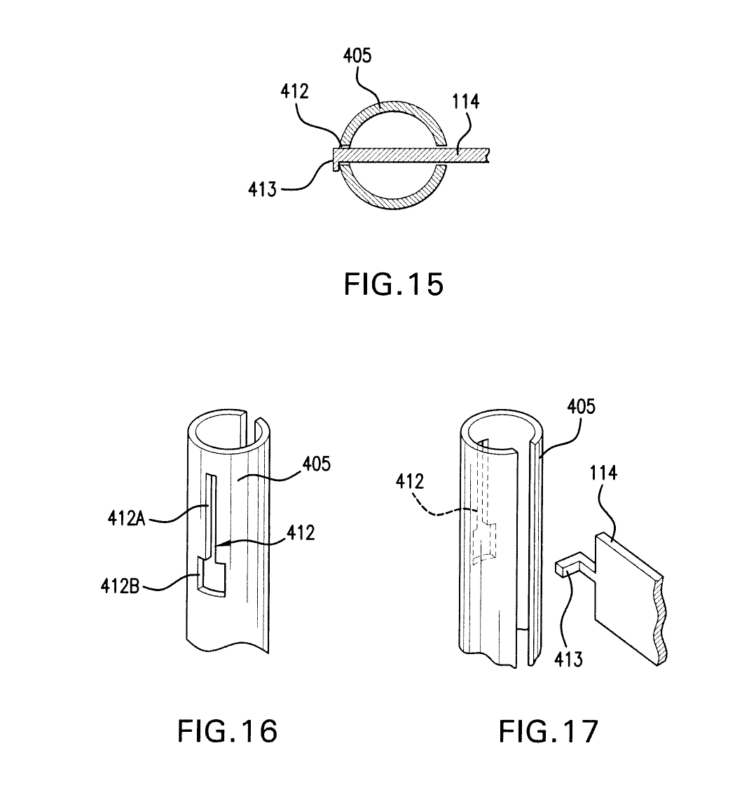

In other embodiments, the shaft portion 405 of the introducer 140 includes a substantially longitudinal opening 412, as shown in FIGS. 15-17. The sensor 114 can include a flange 413 disposed along an edge of the sensor body 114 to communicate through the longitudinal opening 412. The engagement of the longitudinal opening 412 and the flange 413 provide an interference fit to retain the sensor 114. In some embodiments, the slot includes a distal section 412B configured to be wider than the width of a proximal section 412A, and sufficiently wide such that the sensor flange 413 may be displaced from the shaft when the flange becomes disposed in the wider section of the opening 4 12, for example during the insertion process as the sensor travels towards an insertion position. In this manner, the longitudinal opening 412 can be provided with a greater width at a distal section to allow the introducer 140 to be completely de-coupled from the sensor 114 retained within the shaft portion 405 during the placement thereof, so that the introducer 140 may be removed completely from the user, while leaving in place the sensor 114.

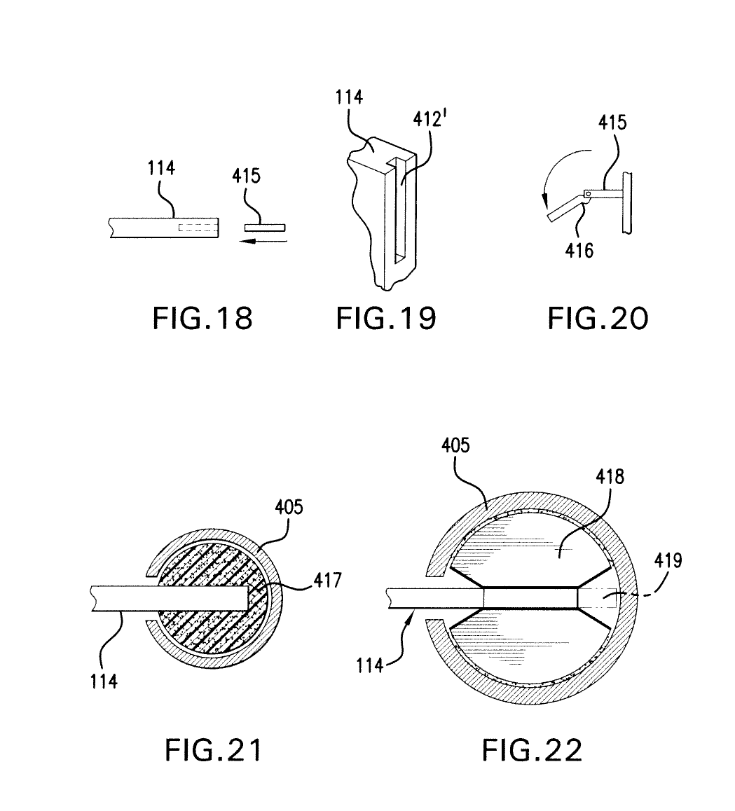

As an alternative, illustrated in FIGS. 18-20 the sensor 114 can be configured to include a pin 415 extending from a lateral end of the sensor body. Similar to the flange member described above, the pin can engage a slot 412′ formed in the introducer so as to retain the sensor in the introducer. In some embodiments, the pin can be configured as a hinge member 416.

In yet another embodiment, the holding member can include a sponge material 417 disposed along the channel of the shaft portion 405, as shown in FIG. 21. The sponge material can be configured to provide a soft interference fit with a sensor 114 disposed in the shaft portion 405 and may comprise polyurethane, polyether, polystyrene, or isoprene foams. The foams can be attached via adhesive, or applied during the lubricious coating process (i.e., a silicone coating used to reduce friction and make insertion more smooth),

In other embodiments, the shaft 405 is provided with a diaphragm 418, such as a thin, semi-rigid membrane housed along a portion of the channel. The diaphragm can include an opening 419 to receive and retain the sensor, as shown in FIG. 22. The diaphragm 418 may be molded or cast polymer (silicone, urethane or TPE) plug or insert with a series of slits or webbing similar to an iris. Or it could be type of a duckbill valve. In one embodiment, the diaphragm 418 is fixed (molded or glued) to the inner diameter of the introducer. The diaphragm 418 may be rigid enough to hold the sensor but flexible to open when the senor is captured during insertion.

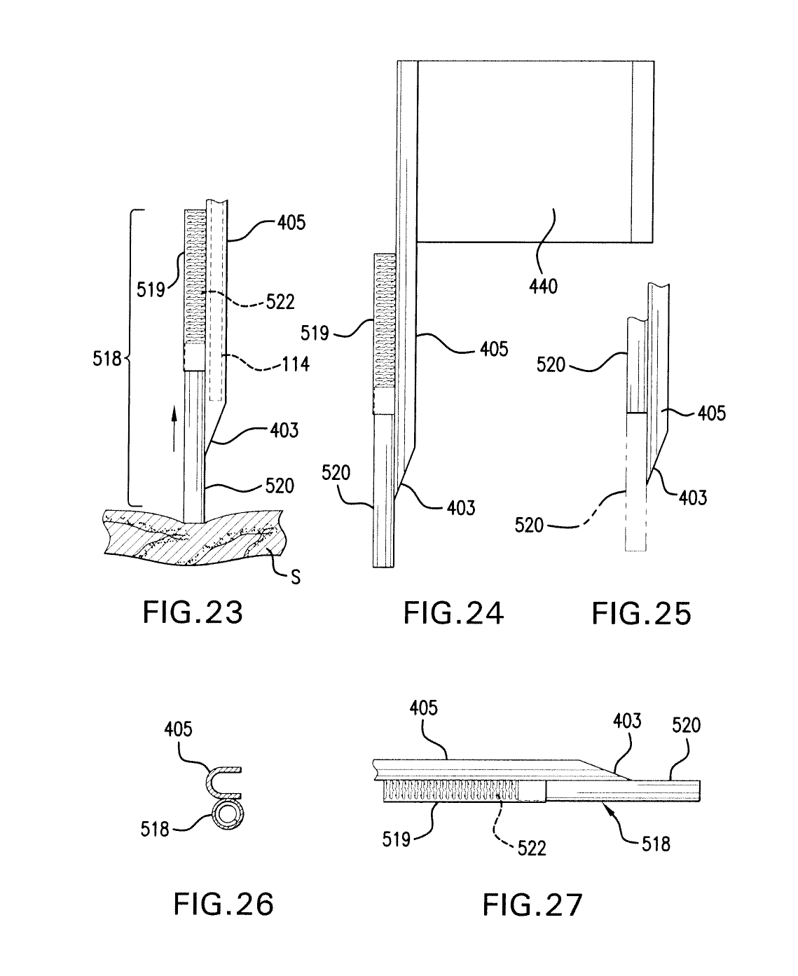

In another aspect of the invention, the introducer 440 may be configured to reduce the insertion and extraction forces through the user's skin, thus reducing trauma to the skin. In this regard, the introducer 440 can be configured to include a compressible member 518 attached to a lateral side of the introducer 440, as illustrated in FIGS. 23-24. In some embodiments, the compressible member 518 can include a first section, or barrel 519, and a second section, or plunger 520, as shown in FIG. 23. The first section 519 can include a compressible body. For example, the compressible body can include a spring, such as a compression spring 522 (illustrated in dashed lines). In some embodiments, the first section 519 includes a housing comprising the spring. The springs may be helical compression springs having variable pitch and compression rate. The shape of the spring can be straight, hourglass, conical or barrel. Alternatively, a controlled friction can be used to allow a plunger 520 to move inside the barrel 519 at a set force. When the predetermined “break force” is reached, the plunger 520 can move. As illustrated in FIGS. 26-27, the shaft 405 of the introducer 440 is attached in some embodiments to the housing of the compression member 518.

In some embodiments, the second section 520 of the compressible member 518 is non-compressible, but retractable. For example, the second section 520 can be formed from a solid thermoplastic member. The first section 519 can be configured to receive the second section 520. In this manner, the compressible member 518 can be compressed upon retraction of the second section 520 within the first portion housing 519. In this regard, the first and second sections can have a telescoping relationship, such that the sliding engagement of the second member upwardly into the first member causes compression of the compressible member, as illustrated in FIG. 25. A first position of second section 520 is illustrated in dashed line and the second position of the second section 520 is illustrated in solid line. The compression of the compressible member 518 by the retraction of the second member 520 causes the distal edge 403 of the introducer shaft, i.e., the sharp, to contact and pierce through the skin of the user.

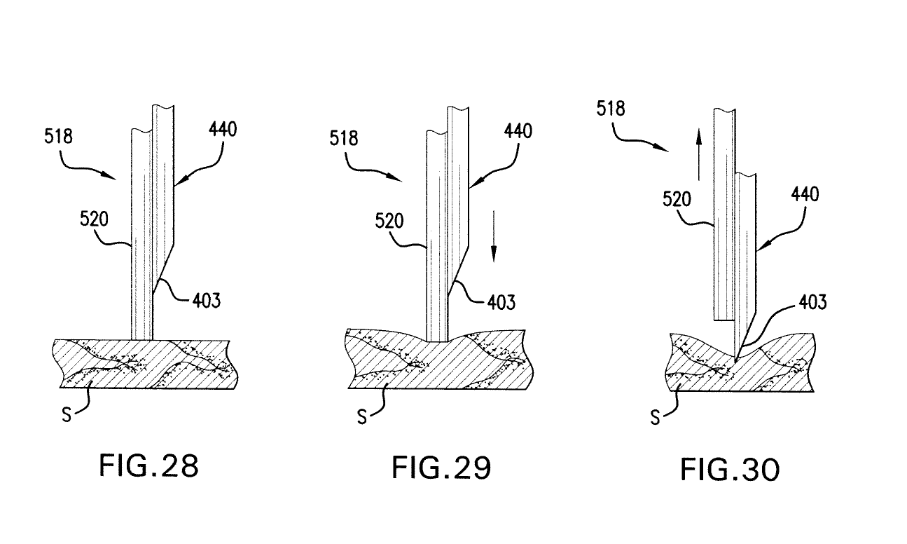

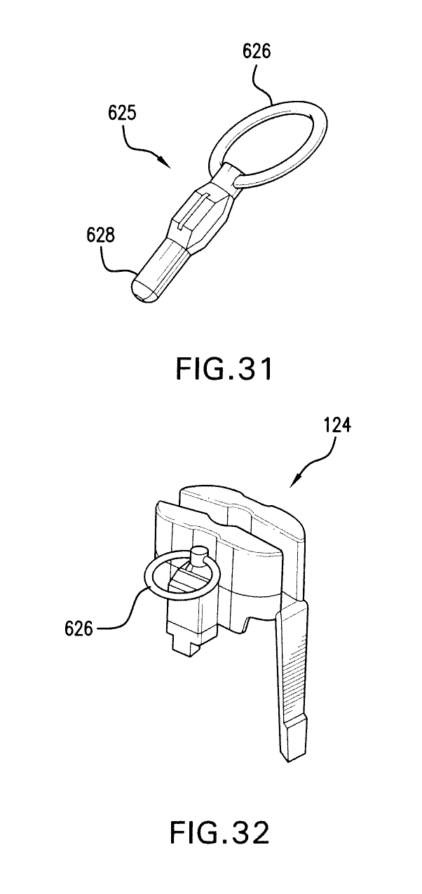

During operation, as shown in FIGS. 28-30, the compressible member 518 contacts the skin S of a user. During this process, the second section 520 of the compressible member contacts the skin S prior to the introducer edge 403 because the distal end of the compressible member 520 is initially distal to the introducer distal end 403. See FIGS. 28-29. In this manner, the second member 520 can tent or depress the skin S from the pressure of the contact between the second section 520 and the skin S. As shown in FIG. 31, the distal end of the introducer 403 then makes contact with the skin S, as the compressible section 518 compressed upon retraction of the second section 520 upwardly to allow the distal end 403 of the introducer 440 to puncture the skin S and proceed to insert the sensor 114 (not shown in FIG. 31). The compressible member 518 allows control of the depth of the puncture. By maintaining a relatively small skin puncture, it is possible to reduce the amount of potential bleeding during the skin piercing process for subcutaneous or transcutaneous sensor placement, and likewise result is less bruising and also faster healing.

In some embodiments, the edge segment 403 of the introducer 440 guides the sensor 114 into and through the skin puncture. The edge segment 403 may be sharpened and polished to facilitate a smooth puncture and a clean cut through the user's skin. In this regard, the substantially hollow shaft portion can be configured to minimize the necessary force to deploy the introducer, and minimize pain and skin trauma during puncture and removal of the introducer from the skin. In this regard, the edge segment 403 of the introducer 440 includes a substantially sharp and angled tip (as shown in FIG. 6) for piercing the user's skin. The edge segment 403 of the introducer 440 can be sharp and tapered to facilitate skin piercing while minimizing skin trauma. In this manner, it is possible to minimize the size of the skin wound at the piercing site where the introducer 440 is placed through the skin, and thus, the user will likely experience a faster healing time.

Referring to FIGS. 31-45, actuator 124 described hereinabove can be provided with a safety member, such as safety member 625, 625′,625″, 634, 636, 650, configured to impede actuation of the actuator, by for example, preventing the actuator button 124 from being depressed. Accordingly, the safety member can avoid accidental firing of inserter assembly 100. The safety member can take the form of various configurations.

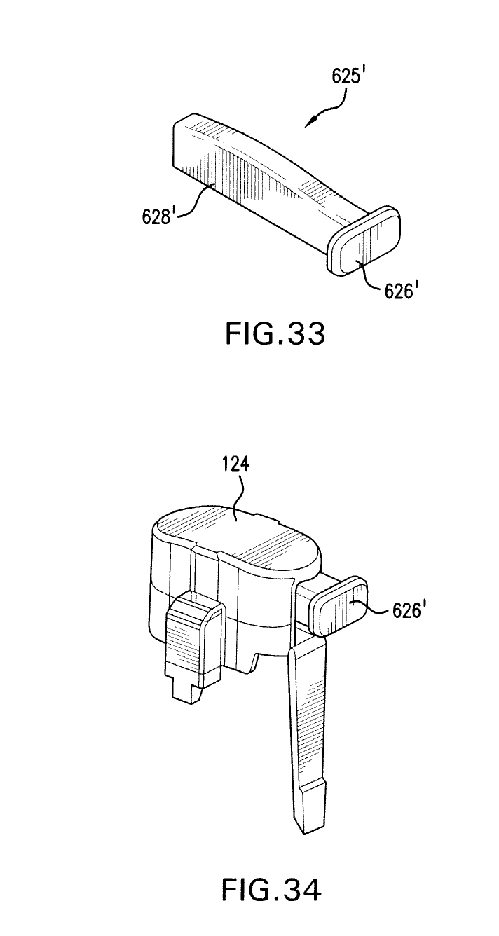

For example, the safety member 625 can comprise a pin or a plug member, such as but not limited to a “grenade” pin, or molded plug, as disclosed in FIGS. 31-36. In this regard, as depicted in FIGS. 31-32, the actuator 124 can include one or more apertures or slots (not shown) extending through the actuator 124 through which the safety pin 628 is disposed. The safety member can further include a pull tab 626 for ease of removal to deactivate the safety. As depicted in FIGS. 33-34, the actuator 124 can include one or more apertures or slots (not shown) extending through the actuator 124 through which the safety pin 628′ is disposed. The safety member can further include a pull tab 626′ for ease of removal to deactivate the safety.

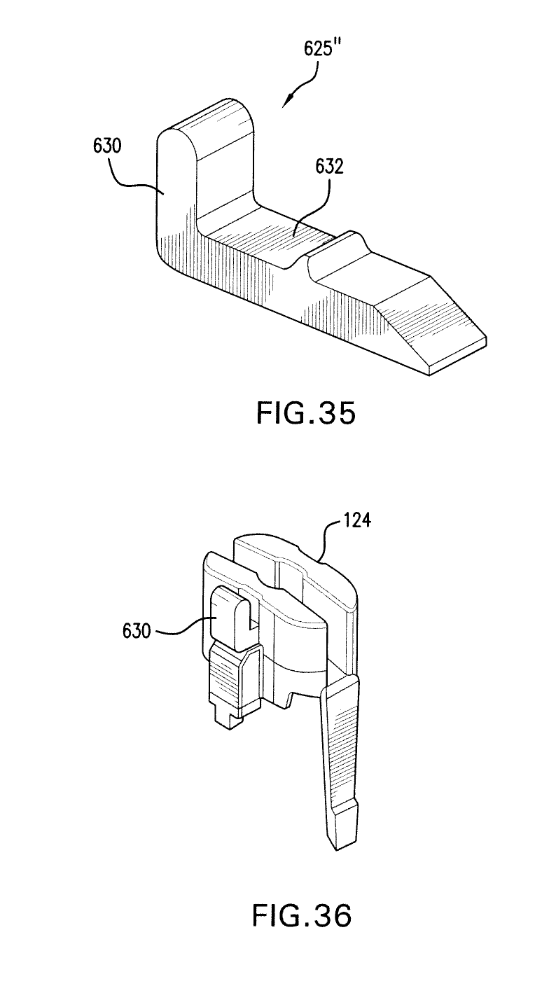

In yet another embodiment, the safety member 625″ can include a body having a first end 630 and a second end 632 configured to form an L-shaped body, as shown in FIGS. 35-36. In this regard, the L-shaped safety member includes, as part of its unitary body a pull tab 630 that protrudes from the slot or aperture formed in actuator 124. In this manner, the first or second ends of the L-shaped body can define a pull tab for deactivation of the safety.

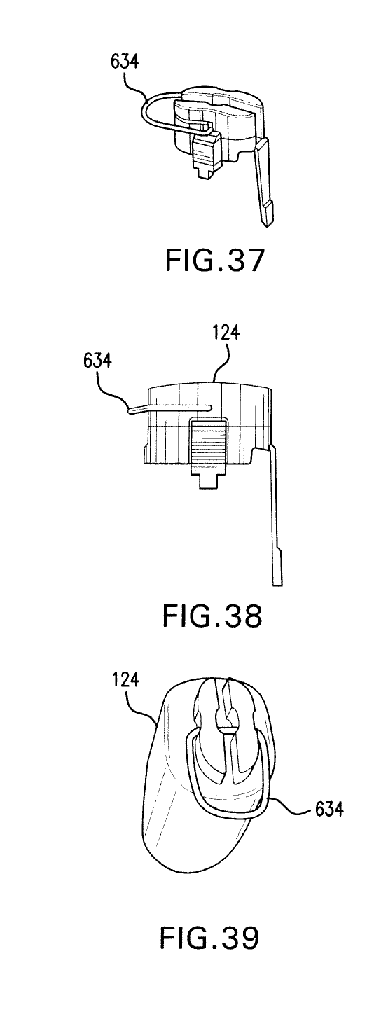

In other embodiments, the safety member comprises a D-ring 634, as shown in FIGS. 37-38. The D ring 634 can be formed from plastic or a metal. As illustrated in FIG. 39, the actuator can include a slot having an opening in communication with the exterior of the actuator. The D-ring can be slid and disposed in the slot, as shown in FIG. 38.

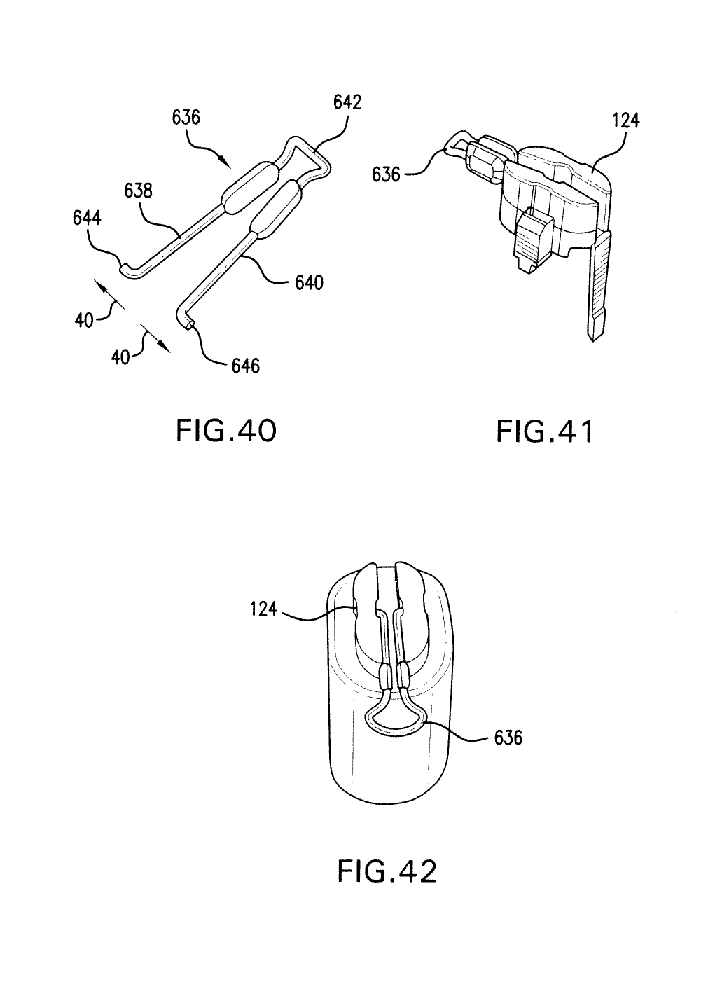

In yet another embodiment, the safety member can comprise a press clip 636, as illustrated in FIGS. 40-42. The press clip 636, in some embodiments, comprises first and second legs 638, 640 connected to each other at a bridging member 642. The press clip 636 includes first and second feet 644, 646 configured to be disposed in one or more apertures formed in the actuator, as illustrated in FIGS. 41 and 42. The configuration of clip 636 provides an outward force, as indicated by arrows 40. The press clip 646 can be disposed in one or more apertures formed on an interior surface of the actuator 124 as illustrated in FIG. 42.

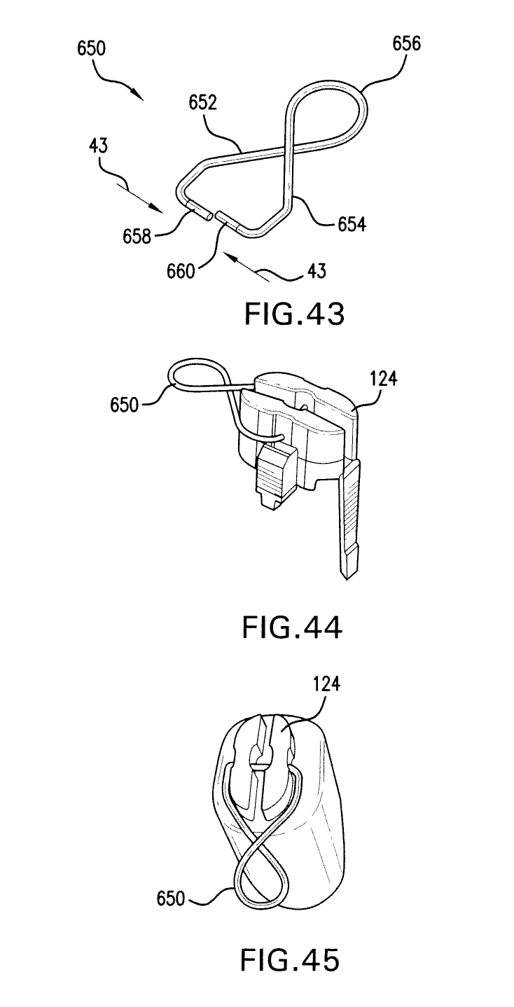

In yet another embodiment, the safety member can comprise a press clip 650, as illustrated in FIGS. 43-45. The press clip 650, in some embodiments, comprises first and second legs 652, 654 connected to each other at a bridging member 656. The press clip 650 includes first and second feet 658, 660 configured to be disposed in one or more apertures formed in the actuator 124, as illustrated in FIGS. 44 and 45. The configuration of clip 650 provides an inward force, as indicated by arrows 43. The press clip 650 can be disposed in one or more apertures formed on an exterior surface of the actuator 124 as illustrated in FIG. 45.

During disposition of the safety member 125 in the actuator, depression of the actuator is impeded. The safety member can be formed from a variety of materials. For example, the material can be a thermoplastic material, such as TPE materials or a metal. In some embodiments, the thermoplastic material has a shore hardness of about 40 to 50. In another embodiment, plastic, metal, wood, or paper can be formed in the shape of a pin as long as it could serve to prevent the downward movement of the button

Upon deactivation of the safety member such as by removal of the safety member, 122, as illustrated in FIGS. 1 and 5, can be squeezed inward just enough to clear the rim 204 of opening 176 while pressing the actuator button 124 down to fire the inserter. Alternatively, tabs 122 can be squeezed further inward so that barbs 206 on the inside edges can engage catches 208 located on a center portion of actuator button 124 by simply pressing down on the actuator button 124.

Referring back to FIG. 5, shuttle 138 is provided with laterally extending barbed fingers 212 which travel in channels 180 along the inside walls of housing 134. When shuttle 138 is inserted up into housing 134 far enough, barbed fingers 212 momentarily deflect inward and then snap outward again to catch on stops 186. In this armed or cocked position as shown, drive spring 136 is compressed and urging shuttle 138 towards base 144, but barbed fingers 212 catching on stops 186 prevent such travel.

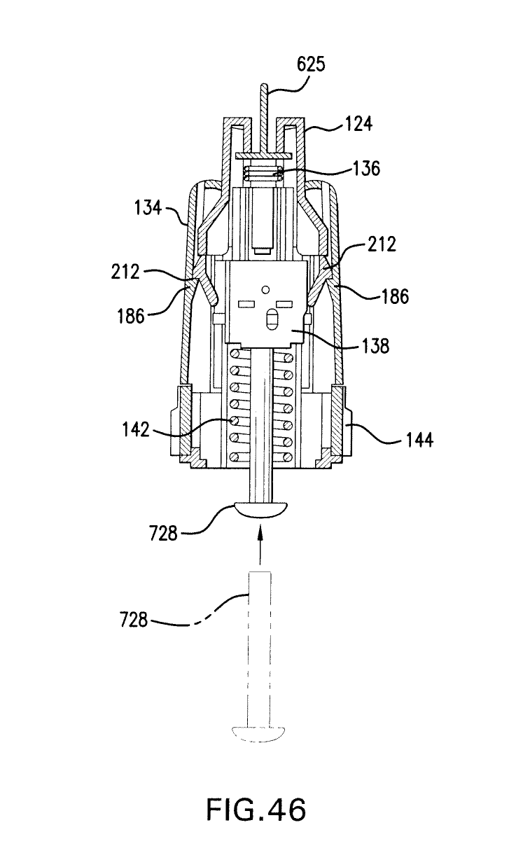

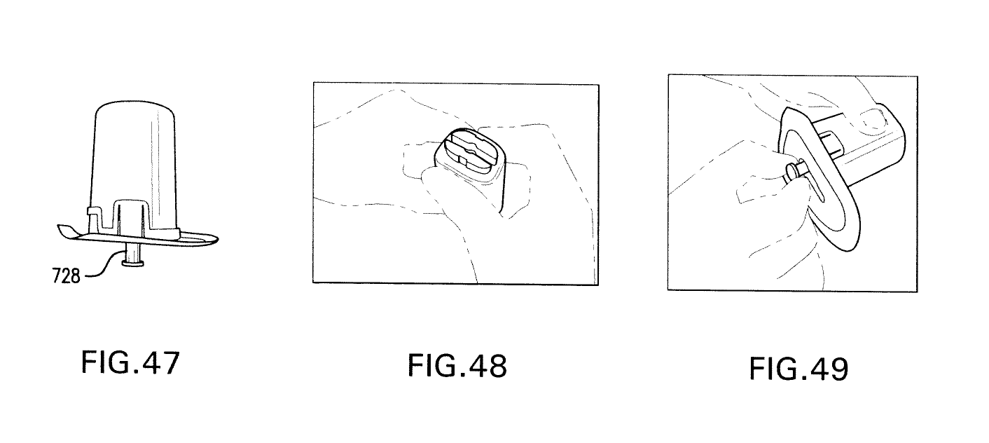

After manufacture of the sensor inserter assembly, the sensor inserter assembly can be shipped in an unarmed position. In this manner, no safety member as described above is necessary for safe shipping or handling as the sensor inserter assembly in its unarmed position cannot fire. In this regard, as shown in FIG. 46 the sensor inserter assembly 110 in its unarmed position can include a pin 728 member, such as a plastic tubular member, disposed in the firing path of the inserter. The pin 728 is configured to butt against the bottom of the shuttle 138 and protrude from the bottom surface of the sensor inserter assembly, as shown in FIGS. 46 and 47. The pin 728 can keep the shuttle from bouncing on the return spring.

In another aspect of the invention, a method is provided to arm the sensor inserter assembly. The sensor can be armed by the user prior to insertion of a sensor. The method includes, as shown in FIGS. 47-49, contacting the sensor inserter assembly against a surface, such as a table top. The contact of the pin 728 with a relatively hard surface causes the pin to be pushed upwardly the retraction position such that the barbed fingers 212 are moved to a cocked position, as described above. In this manner, the sensor inserter assembly can be configured such that an audible click is sounded when the barbed fingers move to position. During movement to the armed position, the actuator button 124 moves upwardly to the cocked position. After the barbed fingers and the actuator are armed, the pin 625 is removed from the sensor insertion assembly and the sensor inserter assembly is armed and ready to use.

In operation, the user arms the drive mechanism, such as the first spring, to generate the sufficient inertial force needed to drive the introducer and the sensor through the user's skin. In one embodiment, the introducer and the sensor are provided in a fully assembled in the sensor inserter assembly package within a transmitter mounting unit. Thus, when the user wishes to place the sensor subcutaneously or transcutaneously, the drive mechanism is armed and the user places the transmitter mount on the surface of the user's skin where the user wishes to place the sensor.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims Additional detailed description of embodiments of the disclosed subject matter are provided in but not limited to: U.S. Pat. No. 7,299,082; U.S. Pat. No. 7,167,818; U.S. Pat. No. 7,041,468; U.S. Pat. No. 6,942,518; U.S. Pat. No. 6,893,545; U.S. Pat. No. 6,881,551; U.S. Pat. No. 6,773,671; U.S. Pat. No. 6,764,581; U.S. Pat. No. 6,749,740; U.S. Pat. No. 6,746,582; U.S. Pat. No. 6,736,957; U.S. Pat. No. 6,730,200; U.S. Pat. No. 6,676,816; U.S. Pat. No. 6,618,934; U.S. Pat. No. 6,616,819; U.S. Pat. No. 6,600,997; U.S. Pat. No. 6,592,745; U.S. Pat. No. 6,591,125; U.S. Pat. No. 6,560,471; U.S. Pat. No. 6,540,891; U.S. Pat. No. 6,514,718; U.S. Pat. No. 6,514,460; U.S. Pat. No. 6,503,381; U.S. Pat. No. 6,461,496; U.S. Pat. No. 6,377,894; U.S. Pat. No. 6,338,790; U.S. Pat. No. 6,299,757; U.S. Pat. No. 6,284,478; U.S. Pat. No. 6,270,455; U.S. Pat. No. 6,175,752; U.S. Pat. No. 6,161,095; U.S. Pat. No. 6,144,837; U.S. Pat. No. 6,143,164; U.S. Pat. No. 6,121,009; U.S. Pat. No. 6,120,676; U.S. Pat. No. 6,071,391; U.S. Pat. No. 5,918,603; U.S. Pat. No. 5,899,855; U.S. Pat. No. 5,822,715; U.S. Pat. No. 5,820,551; U.S. Pat. No. 5,628,890; U.S. Pat. No. 5,601,435; U.S. Pat. No. 5,593,852; U.S. Pat. No. 5,509,410; U.S. Pat. No. 5,320,715; U.S. Pat. No. 5,264,014; U.S. Pat. No. 5,262,305; U.S. Pat. No. 5,262,035; U.S. Pat. No. 4,711,245; U.S. Pat. No. 4,545,382; U.S. Pat. No. 5,356,786; U.S. Pat. No. 5,543,326; U.S. Pat. No. 6,103,033; U.S. Pat. No. 6,134,461; U.S. Pat. No. 6,143,164; U.S. Pat. No. 6,144,837; U.S. Pat. No. 6,161,095; U.S. Pat. No. 6,579,690; U.S. Pat. No. 6,605,200; U.S. Pat. No. 6,605,201; U.S. Pat. No. 6,618,934; U.S. Pat. No. 6,654,625; U.S. Pat. No. 6,676,816; U.S. Pat. No. 6,730,200; U.S. Pat. No. 6,736,957; U.S. Pat. No. 6,932,892; U.S. Publication No. 2004/0186365; U.S. Publication No. 2005/0182306; U.S. Publication No. 2006/0025662; U.S. Publication No. 2006/0091006; U.S. Publication No. 2007/0056858; U.S. Publication No. 2007/0068807; U.S. Publication No. 2007/0095661; U.S. Publication No. 2007/0108048; U.S. Publication No. 2007/0199818; U.S. Publication No. 2007/0227911; U.S. Publication No. 2007/0233013; U.S. Publication No. 2008/0066305; U.S. Publication No. 2008/0081977; U.S. Publication No. 2008/0102441; U.S. Publication No. 2008/0148873; U.S. Publication No. 2008/0161666; U.S. Publication No. 2008/0267823; U.S. Publication No. 2009/0054748; U.S. patent application Ser. No. 10/745,878, filed Dec. 26, 2003 and entitled “Continuous Glucose Monitoring System and Methods of Use”, U.S. patent application Ser. No. 12/143,731, filed Jun. 20, 2008 and entitled “Health Management Devices And Methods”; U.S. patent application Ser. No. 12/143,734, filed Jun. 20, 2008 and entitled “Health Monitor”; U.S. Provisional Patent Application No. 61/149,639, filed Feb. 3, 2009 and entitled “Compact On-Body Physiological Monitoring Devices And Methods Thereof”; U.S. Provisional Application No. 61/291,326 filed Dec. 30, 2009, and U.S. Provisional Application No. 61/299,924 filed Jan. 29, 2010; U.S. patent application Ser. No. 11/461,725; U.S. patent application Ser. No. 12/131,012; U.S. patent application Ser. No. 12/242,823; U.S. patent application Ser. No. 12/363,712; U.S. patent application Ser. No. 12/698,124; U.S. patent application Ser. No. 12/698,129; U.S. patent application Ser. No. 12/714,439; U.S. patent application Ser. No. 12/794,721; U.S. patent application Ser. No. 12/842,013; U.S. Patent Application Ser. No. 61/238,646; U.S. Patent Application Ser. No. 61/345,562; U.S. Patent Application Ser. No. 61/361,374; and elsewhere, the disclosures of each are incorporated by reference in their entirety herein for all purposes.

The foregoing only illustrates the principles of the disclosed subject matter. Various modifications and alterations to the described embodiments will be apparent to those skilled in the art in view of the teachings herein. It will be appreciated that those skilled in the art will be able to devise numerous modifications which, although not explicitly described herein, embody the principles of the disclosed subject matter and are thus within the spirit and scope of the disclosed subject matter.

What is claimed is:

1. An introducer comprising:
a body portion having a proximal end and a distal end; and
a shaft portion comprising a channel and a distal end, the shaft portion extending downwardly from an edge of the body portion, the shaft portion including a holding member disposed along a length of the channel, the holding member configured to substantially releasably retain a sensor.

2. The introducer of claim 1, wherein the holding member comprises one or more rolling members disposed along a length of the shaft portion, the rolling members configured to contact and releasably retain the sensor.

3. The introducer of claim 2, wherein the rolling members are configured to rotate.

4. The introducer of claim 3, wherein a sensor retained by the shaft portion may be displaced from the shaft portion upon rotation of the rolling members.

5. The introducer of claim 2, wherein the one or more rolling members are disposed within the channel.

6. The introducer of claim 5, wherein the one or more rolling members are disposed within a sidewall of the channel.

7. The introducer of claim 1, wherein the holding member comprises a sheath disposed coaxially about the shaft portion.

8. The introducer of claim 7, wherein the sheath comprises one or more perforations along a perforation line disposed along a length of the sheath.

9. The introducer of claim 8, wherein the sheath is a tear away member.

10. The introducer of claim 7, wherein the sheath comprises a polymer film.

11. The introducer of claim 10, wherein the polymer film is attached an outer surface of the shaft portion.

12. The introducer of claim 1, wherein the shaft portion comprises a magnet, and further wherein a sensor releasably retained in the shaft includes a magnetic metal.

13. The introducer of claim 12, wherein the magnetic metal comprises stainless steel.

14. The introducer of claim 12, wherein the sensor is doped with a magnetic metal.

15. The introducer of claim 12, wherein the magnet is disposed along the channel of the shaft portion.

16. The introducer of claim 1, wherein the shaft portion includes one or more windings configured to releasably retain the sensor.

17. The introducer of claim 16, wherein the sensor is capable of being displaced from the shaft portion by unwinding the one or more windings.

18. The introducer of claim 16, wherein the windings comprises wound rolls of polymer film.

19. The introducer of claim 1, wherein the shaft portion includes an aperture formed in the channel, and further wherein the sensor includes a flange extending from an edge of the sensor, the flange disposed in the aperture formed in the channel.

20. The introducer of claim 19, wherein the aperture includes a section configured to be wider than the width of a sensor flange such that the sensor may be displaced from the shaft.

21. The introducer of claim 1, wherein the holding member comprises a sponge material disposed along the channel of the shaft portion, the sponge material configured to provide a soft interference fit with a sensor disposed in the shaft portion.

22. The introducer of claim 1 wherein the shaft portion is substantially hollow.

23. The introducer of claim 1 wherein the shaft portion is configured to retain at least a portion of the sensor substantially subcutaneously when the shaft portion is removed from a skin layer.

24. The introducer of claim 1 wherein the distal end includes a tapered end configured to pierce the skin layer, and further, wherein at least a portion of the sensor is substantially retained within the shaft portion while the tapered end is piercing through the skin layer.

25. The introducer of claim 24 wherein when the tapered end of the shaft portion is transcutaneously introduced to the skin layer, the sensor is substantially contemporaneously transcutaneously introduced through the skin layer.

26. The introducer of claim 1 wherein the sensor includes an analyte sensor.

27. The introducer of claim 26 wherein the analyte sensor is a glucose sensor.

28. The introducer of claim 26 wherein the introducer is configured to position an analyte sensor in fluid contact with the user's analyte.

29. The introducer of claim 1, further including a compressible member having a distal end, the compressible member attached to a lateral side of the shaft portion, wherein the distal end of the compressible member is distal to the distal end of the shaft portion.

30. The introducer of claim 29, wherein the compressible member is configured to retract to allow the sharp to penetrate skin of a user.

31. An introducer comprising
a body portion having a proximal end and a distal end;
a shaft portion comprising a channel and a distal end, the shaft portion extending downwardly from an edge of the body portion, and
a compressible member having a distal end, the compressible member attached to a lateral side of the shaft portion, wherein the distal end of the compressible member is distal to the distal end of the shaft portion.

32. The introducer of claim 31, wherein the compressible member includes a first section and a second section.

33. The introducer of claim 32, wherein the first section includes a compressible body.

34. The introducer of claim 33, wherein the compressible body includes a spring.

35. The introducer of claim 34, wherein the spring is disposed within a housing.

36. The introducer of claim 32, wherein the second section is non-compressible.

37. The introducer of claim 32, wherein the first section is configured to receive the second section.

38. The introducer of claim 37, wherein the second section is retractable within the first portion.

39. The introducer of claim 38, wherein the first and second sections have a telescoping relationship.

40. A sensor inserter assembly comprising
a housing;
a shuttle movably connected to the housing, the shuttle movable in an insertion direction and a retraction direction;
a first spring means for urging the shuttle in the insertion direction;
a second spring means for urging the shuttle in the retraction direction;
a sensor;
an introducer attached to the shuttle, the sensor introducer configured to releasably receive the sensor;
an actuator having a depressible body, the actuator configured for releasing the shuttle and allowing the first spring means to urge the shuttle and introducer in the insertion direction, wherein the depressible body includes a safety member to impede the actuator.

41. The sensor insertion assembly of claim 40, wherein the safety member is disposed in one or more apertures formed in the depressible body of the actuator.

42. The sensor insertion assembly of claim 41, wherein the disposition of the safety member impedes depression of the actuator.

43. The sensor insertion assembly of claim 42, wherein the safety member comprises a grenade pin.

44. The sensor insertion assembly of claim 43, wherein the grenade pin includes a pull tab.

45. The sensor insertion assembly of claim 42, wherein the safety member comprises a plug disposed in an aperture formed in the depressible body of the actuator.

46. The sensor insertion assembly of claim 45, wherein the plug includes a pullable cap disposed at and end of the plug.

47. The sensor insertion assembly of claim 40, wherein the safety member has a body including a first end and a second end, and further wherein the first and second ends form an L-shaped body.

48. The sensor insertion assembly of claim 47, wherein at least one of the first or second ends is disposed in an aperture formed in the actuator.

49. The sensor insertion assembly of claim 48, wherein the other of the first or second ends forms a pull tab for deactivation of the safety.

50. The sensor insertion assembly of claim 40, wherein the safety member includes a D-ring.

51. The sensor insertion assembly of claim 50, wherein the actuator includes a slot to slidably receive the D-ring.

52. The sensor insertion assembly of claim 40, wherein the safety member includes a press clip configured to be received in one or more aperture formed on the actuator.

53. The sensor insertion assembly of claim 52, wherein the press clip includes a first leg and a second leg.

54. The sensor insertion assembly of claim 53, wherein the first leg is disposed in an aperture formed in a first side of the actuator depressible body, and the second leg is disposed in an aperture formed in a second opposite side of the actuator depressible body.

55. The sensor insertion assembly of claim 40, wherein the safety member is formed from a molded thermoplastic material.

56. The sensor insertion assembly of claim 55, wherein the thermoplastic material has a shore hardness of about 40 to 50.

57. A method of arming a sensor inserter assembly comprising a shuttle movable in an insertion direction and a retraction direction, a first spring means for urging the shuttle in the insertion direction, a second spring means for urging the shuttle in an opposite retraction direction, an introducer having a sensor disposed therein, the introducer attached to the shuttle, an actuator configured for releasing the shuttle and allowing the first spring means to urge the shuttle and introducer in the insertion direction, and an arming pin disposed on the method comprising:
contacting the distal end of the arming pin on a surface until the actuator moves to an upward position; and
pulling pin from the sensor inserter assembly to remove the pin.

58. The method of claim 57, wherein the method includes contacting the distal end of the arming pin with a surface until the actuator clicks.

59. The method of claim 57, wherein no safety member is required.
